(19) United States
(12) Patent Application Publication
(10) Pub. No.: US 2017/0274149 A1
(43) Pub. Date: Sep. 28, 2017

(54) Injection System

(71) Applicant: MEDAXOR PTY LTD, Scoresby, Victoria (AU)

(72) Inventor: Andreas Aeschlimann, Scoresby, Victoria (AU)

(21) Appl. No.: 15/508,482

(22) PCT Filed: Aug. 30, 2015
(86) PCT No.: PCT/IB2015/056569

§ 371 Date: Mar. 2, 2017

(30) Foreign Application Priority Data
Sep. 8, 2014 (AU) 2014903561

(51) Int. Cl. A61M 5/315; A61M 5/31; A61M 5/20

(52) U.S. Cl. CPC A61M 5/31568; A61M 5/20; A61M 5/3146; A61M 2205/50; A61M 2205/33; A61M 2205/3592; A61M 2205/3584; A61M 2205/8206; H04W 4/008

(57) ABSTRACT

The present invention provides apparatus and systems for enabling and monitoring injection of medicinal fluids into a subject. The invention includes a housing for a prefilled syringe, the housing incorporating means for generating and sending signals as parts of the syringe cooperate to deliver the fluids. The signals may be transmitted from a transmitter on the housing as radiowaves to a receiver, which may be conveniently located on a spaced device, such as a mobile phone. The mobile phone or other receiving device incorporates computer software to receive, store the signals from the transmitter as a record of the injection process. The computer software may include information about the medicine, the injection process, and other information relevant to a treatment regime. In some cases, the receiving device may transmit information stored by the computer software for storage and analysis.

### TECHNICAL FIELD

The present invention relates to fluid delivery devices and systems, in particular, medical syringes, and more particularly, medical syringe pens and systems for self-injection.

### RELATED APPLICATION

This application claims the priority and benefit of Australian provisional application 2014903561 Sep. 8, 2014.

### BACKGROUND ART

Many pharmaceutically active substances must be delivered as injectable solutions using a hypodermic needle and syringe. In recent years, self-injecting systems or self-injection pens have become more popular. A shift to pre-filled, disposable, self-injection devices aims to address convenience, regulatory compliance requirements, and needle phobia/patient compliance. Such systems are rapidly expanding across therapeutic areas such as devices for the treatment of multiple sclerosis (MS), rheumatoid arthritis (RA), fertility, osteoporosis, hepatitis, oncology, anaemia and migraine headaches.

Traditionally, a self-injection pen is designed around a primary drug container in the form of a pre-filled syringe or cartridge. Self-injection devices such as “pen injectors” and “auto-injectors” act as the secondary packaging for the primary drug container (cartridges or prefilled syringes).The device is designed to simplify the drug administration process for the user. Some of the key characteristics are: usability, end-of-dose indication, automatically activated needle shields preventing sharps injuries, and visual indication that dose has been fully delivered.

The primary drug container, prefilled syringe or cartridge, in which the substance is contained, is filled with automated aseptic filling lines. After filling and closing the primary container with the appropriate closure system there is a small amount of air contained in the primary drug container. This air has to be removed from the primary drug container by the user before an injection can be given.

A new user on a new treatment plan needs to be aware of the timing and amount of medicine to use. For example, a program involving daily, or even more frequently, injections of insulin are often necessary for diabetes sufferers. A user also wants to minimise the chance that a needle stick injury might occur when handling a self-injection system. To eliminate the need for drawing up from a vial or reconstituting from two vials, medicine manufacturers often develop pre-filled syringes of medicines to help with this problem.

Pharmaceutical companies are also reformulating some substances in order to optimise the frequency of injection. This is particularly true in the case of non-chronic diseases, or when the treatment regimen dictates infrequent drug administration (e.g., once every 2 or 4 weeks). In such cases the user might be less familiar with the self-injection device and may have to refer to the instruction manual for use every time the user is due for an injection.

Existing self-injecting devices do not offer an integrated interface to digital health databases enabling automatic control of prescribed therapy regime or improved patient engagement with the digital health systems. In many cases end-of-dose delivery indication is cumbersome and generally a user or patient has to countdown to ensure a full dose has been delivered from the syringe.

Many self-injection devices require multiple administration steps and are therefore complicated to use. In particular, dual-chamber injection pens where mixing of lyophilised drug and diluent and priming is required can have multiple administration steps.

What is needed is a commercially viable and compact communication interface between a primary drug container of an injection system and a mobile communication device improving the usability and convenience factor of the injection system device.

### SUMMARY OF INVENTION

The present invention provides apparatus and systems for delivering aqueous fluids, in particular, medicines and other therapeutic fluids, by injection. In one aspect the invention provides a housing, a pre-filled syringe, and a control system for enabling injection and monitoring the injection process. Alternatively the apparatus described herein may be used in combination with other primary drug container of an injection system such as a wearable injector, infusion pumps, auto injectors and the like. The invention provides an apparatus to generate signals at predefined stages of the injection process. The signal sequencing is used to trigger appropriate prompts and commands within the app and allows to record details such as end of dose delivery etc.

The invention very advantageously provides a monitoring and guidance system for delivering substances by injection. Most advantageously, the invention system provides means to guide a user when to inject substances according to a predefined, but modifiable, program, such as for therapeutic drug injection, and monitors that the user has made the injection according to a treatment plan. Most advantageously, the program can be modified offline and remotely by a medical practitioner to best treat an indication using the invention by using the injection system's communication interface with a telecommunication device. The injection system obviates or minimises the need for a user manual to be consulted by a user self-injecting, particularly when commencing a program of injections needed only infrequently, say, once per week.

In recent years wireless communication technology has become popular but has failed to be used in self-injection devices possibly due to its cost, limitation in size and power consumption. Most recently, mobile operating systems such as Android and iOS have included to support new and further developed wireless communication protocols such as Bluetooth low energy (BLE), Near Field Communication (NFC) and ANT. The invention most advantageously exploits these technologies by utilising the screen of a telecommunication device as a digital and interactive instruction means for use with an injection pen. Further, the invention incorporates a communication interface which enables the automated control and monitoring of a prescribed therapy regime and helps to improve patient engagement and provide online user feedback.

In one aspect, the invention provides an injection pen comprising of a housing for a syringe having a plunger, a plunger rod for activating the plunger, and communication means for communicating the displacement of the plunger. The injection pen may include a cap.

In another aspect, the invention provides an injection pen system comprising of: an injection pen including a housing for a syringe, and syringe, means for operating the plunger of said syringe, communication means for transmitting and receiving signals; and a microprocessor programmed with computer software for monitoring and guiding an injection from the syringe.

The injection pen may incorporate an antenna for sending signals to a receiver. Preferably the signals are sent and received using radiofrequency means. Preferably the signal data is transmitted by an RFID chipset. Preferably, the power source for transmitting signals is a battery. Preferably, the power source and the communication means are interchangeable. Preferably the signals are received by a mobile phone. Preferably, the mobile phone incorporates computer software for analyzing the received signals into information for transmission to record and further analyse the injection data.

### BRIEF DESCRIPTION OF DRAWINGS

FIG. 1 shows in planar view the main elements of an embodiment of an injection pen system according to the invention.

FIG. 2 shows in planar view an exploded embodiment of an injection pen according to the invention.

FIG. 3 shows in planar view an antenna for communicating and generating signals and an antenna sleeve for locating the antenna and an embodiment showing an alternative method for generating signals

FIG. 4 shows planar views of embodiment of plungers which may incorporate an electronic chipset with integrated switch for communicating the status of an injection pen according to the invention

FIG. 5 shows in planar view an embodiment of a device integrated electronic chipset and battery casing for an injection system and an embodiment of an external electronic chipset in a casing.

FIG. 6 shows in planar view and embodiment of a release and drug container priming mechanism for an injection pen according to the invention.

FIG. 7 shows an embodiment of an injection pen system including a schematic representation of the communication elements, including external chipset, integrated switch, external antenna.

FIG. 8 shows the steps taken to carry out an embodiment of the invention

### DESCRIPTION OF THE PREFERRED EMBODIMENTS

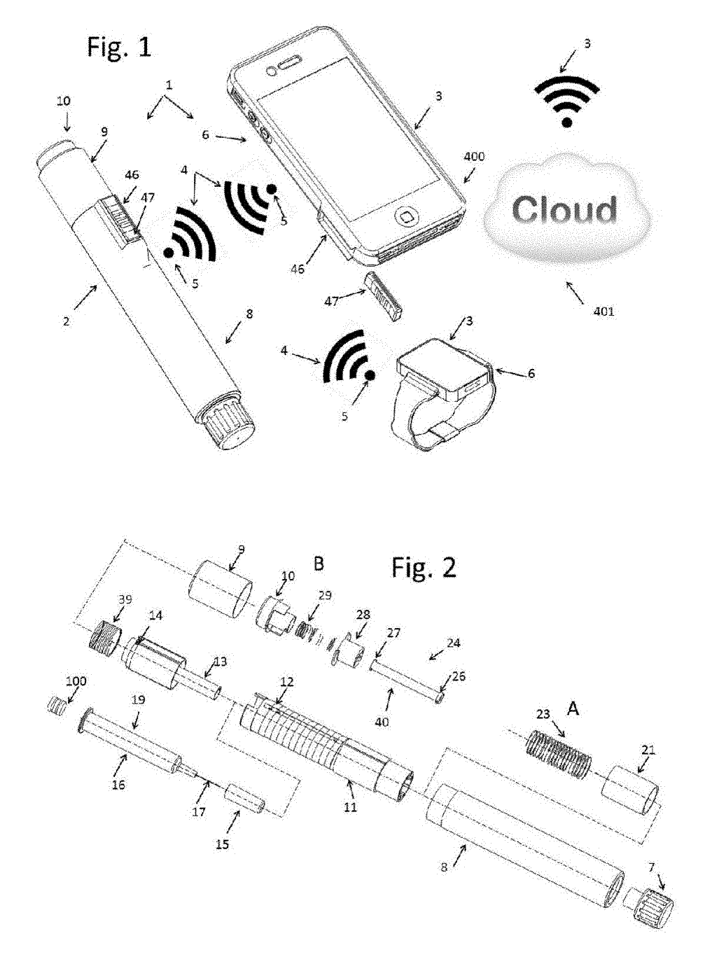

The invention is best understood by the description herein and associated drawings in the figures. It will be understood that the scope of the invention includes embodiments not shown in this disclosure and that the scope of the invention is limited only by the claims appended hereto. An embodiment of the invention is shown in FIG. 1, where an injection pen system 1 according to the invention incorporates an injection pen 2, mobile communication device 3 including a microprocessor 6 for transmitting and receiving data from the injection pen 2. Both the injection pen 2 and mobile communication device 3 incorporate radio waves/signals 4 received and transmitted by an internal antenna for transmitting and receiving data. The injection pen 2 may or may not incorporate a power source for powering its antenna. Power may be provided remotely, such as with radio waves. In this embodiment, the mobile communication device incorporates a battery for a power source. In embodiments including a battery, the invention may provide a method for external chipset and battery management. The external electronic unit 47 can be removed from the injection system and inserted into the casing 46 of the mobile telecommunication device, (mobile phone in this illustration) until the next injection with a new single use injection system. Further, in embodiments including the external electronic unit, it also provides a method for discarding the electronic unit separately which helps to reduce the environmental impact of the invention. Preferably, the mobile communication device 3 is a mobile telephone or cell phone. Alternatively, the mobile communication device may be a watch or other device that may be worn on the wrist, for example.

The invention includes a computer software program, which is preferably disposed in the telecommunication device, for interactively directing a user in carrying out an injection procedure, and collecting and storing data related to the procedure. This includes, without limitation: the time, the place, injection volume, the injection site on the body, as well as information about the therapeutic substance in the primary drug container, such as the active compound, the manufacture date, the batch number, and the expiry date which may be programmed into a device internal chipset. Other data may be collected in some embodiments. This data is embodied in the signals transmitted by the antenna and automatically captured by the communication between the injection pen and the software application. Further, an advantage of loading the software on a mobile communication device such as mobile phone is that the data can be transmitted to various recipients, such as of the practitioner prescribing the therapeutic treatment, the manufacturer of the medicament, or the controller of a clinical trial. The mobile phone application allows engagement with a user database of injections, frequency, and therapeutic treatment substances, concentrations, and the like.

The invention most conveniently may include post-market surveillance, for example, by a drug manufacturer, and makes it easy to contact a drug user by the manufacturer in the case of a recall, for example. The drug manufacturer could conveniently recall a certain lot number in a production database and the end-user would be notified and warned should he attempt to inject a recalled drug/lot number. Since a number of parameters are recorded about each injection procedure, it conveniently allows the device user to direct input user feedback via the software interface to the drug development department of a drug supplier so that user feedback can be collected more efficiently, possibility prompting modifications to a next generation injection device, or therapeutic program, for examples. Connecting the end-user with the drug and device developer can improve the product development cycle and can fast-track the administrative tasks within a clinical evaluation of such a drug/device combination.

The invention provides a mechanism to automatically prime (expel air) from the primary drug container and provide instance feedback to the user after priming is completed via the GUI of the mobile communication device.

Further, the invention provides a system to externally handle the electronic chipset 47, including battery 112, casing 400 for telecommunication device with integrated housing 46 and an injection system with integrated housing 46 so that the chipset unit can be conveniently transferred to other single-use pens or removed and discarded appropriately. The external electronic unit may be pre-inserted in the injection system as a default configuration. The electronic unit 47 is removed and placed into the casing 400 after the injection has been completed. The electronic unit may also contain a NFC chip which can be read by the telecommunication device, ensuring that the electronic unit 47 is stored in place and ready to be used for the next injection.

The data collection by the manufacturer may help to shorten the innovation cycle in developing safe and efficacious drugs and devices for treatment.

Injection System

FIG. 1 shows an embodiment of elements of a system and apparatus for guiding and monitoring the injection of fluids using an injection pen incorporating a primary drug container, preferably a pre-filled syringe of medicine. The embodiment incorporates a delivery device 2 in communication with a mobile communication device 3, which, in turn, may be in communication with databases and stakeholders in providing diagnosis and health benefits 401. Most advantageously, the delivery device is an injection pen for primary drug containers, preferably a pre-filled syringe and communication exploits the speed and bandwidth of the internet to acquire, store, and access data and information. FIG. 1 also shows a casing for a mobile communication device 400 with integrated housing 46 which can receive and hold the external electronic unit 47. The delivery device 2 is shown with the external electronic unit inserted in the pocket of the pen housing 8. The external electronic unit 47 can be removed after the delivery device has been used and inserted back into the housing 46 integrated in the casing 400 of the mobile communication device 3. FIG. 1 shows that the mobile communication device 3 may take any convenient form such as a mobile telephone or a watch.

Injection Pen

An embodiment of the injection pen 2 component of the invention is shown assembled in FIG. 1 and in exploded view in FIG. 2. FIG. 1 shows the assembled injection pen comprises of a pre-filled syringe housing 8 with incorporated slot for external electronic unit 47 engaged with an end housing 9. A cap 7 engaged at one end of the syringe housing is preferably press fitted with the cap 15 of the pre-filled syringe which protects the needle tip of the syringe and from needle stick injuries. A button 10 at the other end of the injection pen covers the injecting mechanism and enabling the user to initiate the drug injection. The primary drug container housing 8 encloses an antenna sleeve 11 shown in FIG. 2.

FIG. 2 shows the components of the injection pen 2 in exploded view. The dashed lines indicate which components are located within which other components. Components are arranged generally concentrically and along the axis of the injection pen 2. A primary drug container such as a pre-filled syringe 19 is conveniently inserted into the syringe housing 8 of the injection pen 2. Insertion of a primary drug container, preferably a pre-filled syringe 19 known in the art with its barrel 16 pre-filled with an appropriate medicinal solution for injection using the injection pen. The components of group A and group B cooperate to inject liquid from the drug container barrel 16 through the needle 17 of the syringe 19 when the springs of the injection pen are released. Housing 8 may contain cut-outs so that the liquid stored in the primary drug container can be inspected prior its use.

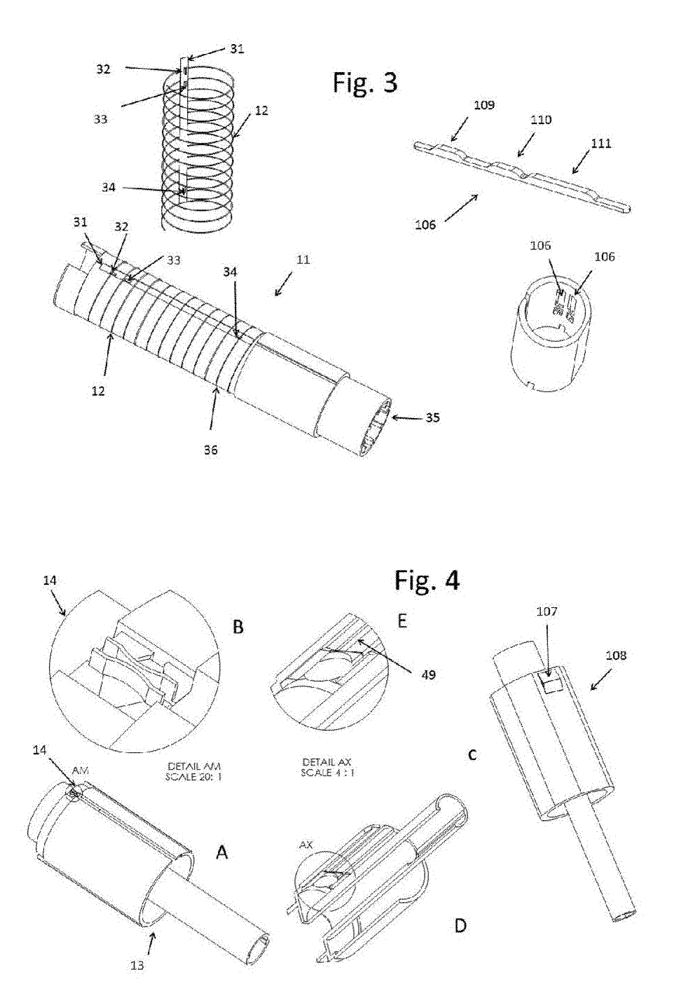

Inside the pen housing 8 is the antenna sleeve 11 around which is wound an antenna 12 for transmitting signals 4 of the status of the injection process. The antenna sleeve 11 is inserted with a switching plate 31 allowing making contact with the counterpart 14 shown in FIG. 4. Alternatively the antenna sleeve 11 may contain an alternative switching plate 106, arrayed in parallel shown in FIG. 3 making contact with counterpart 107 incorporate in plunger 108 shown in FIG. 4. In case of the alternative switching plate 106, the antenna 11 may be incorporated in the electronic unit 118.

Within the syringe housing 8 at the cap end 7 are the group of components A biased toward the cap end 7. This group of components A is located within the housing 8 when the injection pen 2 is in the closed position as shown in FIG. 1. The group of components A in FIG. 2 may include a needle protector 21 known in the art to protect and guide the operation of the pre-filled syringe 19 and its needle 17 during the injection process. The protector 21 may be biased with a spring 23 in some embodiments. The group of components A fit within the housing 8 for the syringe.

The group of components B in FIG. 2 fit within the antenna sleeve 11 and plunger 13 of the primary drug container 19. The components B provide a mechanism for automatically priming and initiating the injection of the substance. Plunger rod 24 refrains the plunger 13 from going forward (injecting the drug) after priming is completed by the lugs 27 and shoulder 26 of plunger rod 24. The force for biasing the plunger 13 of the primary drug container 19 to force liquid through the needle 17 during an injection is provided by spring 39. A button 10 within the end housing 9 of the injection pen is inserted with a button spring 29 within the end housing 9 biases the button 10 toward the housing 9. The button spring 29 biases the button 10 against the end of the housing 9 while the releasing ring 28 is in the closed position. The release ring 28 is rotated to the open position by pushing the button 10. When button 10 is pressed down, the release ring 28 is rotated releasing the lugs 27 of plunger rod 24 and enabling the plunger 13 to move forward pushing piston 100 down the barrel of the primary drug container 16. The plunger 13 then pushes against piston 100 of the syringe 19 to inject liquid.

FIG. 4 shows three embodiments of plungers A-D. One embodiment D incorporates an internal power source 49. Another embodiment C incorporates a plunger used in combination with an external power source 49.

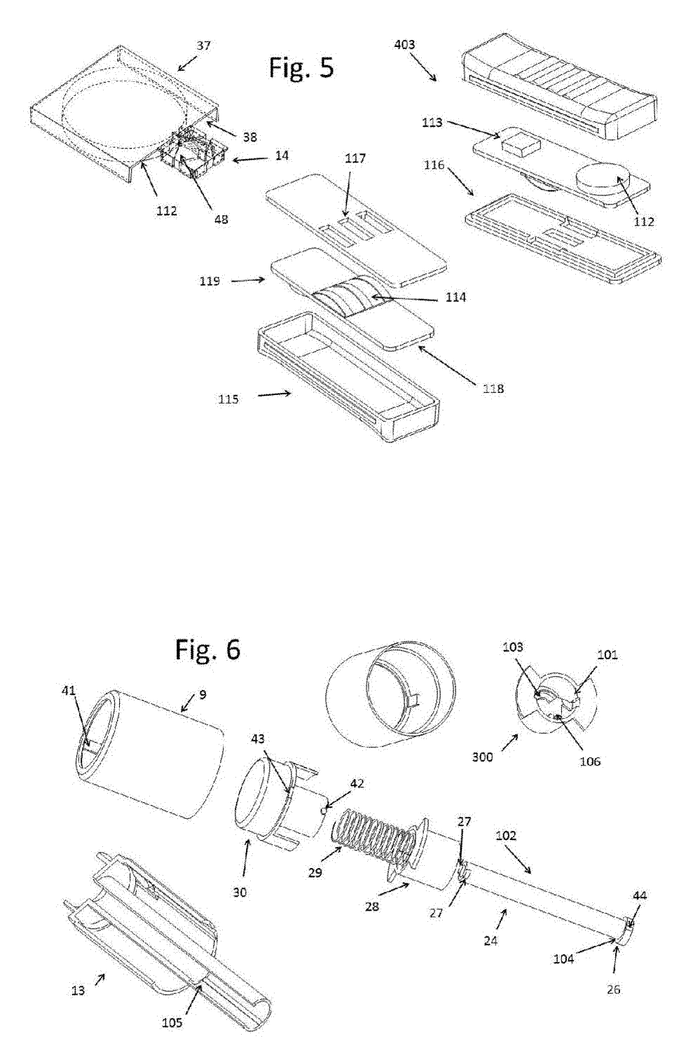

FIG. 5 shows an electronic chipset 14, which may include a wireless communication stack, micro-processor, with integrated contact 48, a power source, preferably a battery 112 and battery housing 38 and an embodiment of an external electronic chipset 118, preferably including a battery 112, microprocessor 113, on-board contact points 114 and wireless communication elements. It may also include a NFC chip. The battery 112 may be recharged via the mobile phone 3 while inserted into casing 400. The chipset 118 is housed in casing 119. The casing 119 consist of at least a cover 116 and housing 115. Cover 116 includes cut-outs 117 for allowing the on-board contacts 114 to protrude trough the cover 116 and making contact with the contacts housed in the injection system 2. Preferably the multiple contacts 114 will be used to power the chipset 118 when inserted into housing. Advantageously, the casing 119 design is symmetrical and can therefore be inserted either way into housing 46 of pen housing 8 and casing 400 of mobile communication device 3. In this embodiment, the cover 115 has a rib 403 which runs along the outside which conveniently keeps the casing 119 in place when inserted. Alternatively the unit 118 may be hold in housing 46 with a light press fit.

FIG. 6 shows the components of an embodiment of the release mechanism shown in FIG. 2 in more detail. The plunger rod is spaced from the plunger. Preferably there is 5-10 mm (depending on the amount of air to be expelled) between the face 104 of plunger rod 24 and face 105 of plunger 13 in the injecting pen's default positon. In this position the device integrated switch 48 is active and can transmit a signal to the mobile communication device 3 via the device integrated wireless communication system 5. The priming is initiated by removing cap 7 of the pen 1 which is preferably press fitted with cap 15 of the syringe. By removing cap 15 of primary drug container 16 plunger 13 will be pushed forward by the spring 39 until face 105 of plunger 13 meets face 104 of plunger rod 104. Moving plunger 13 to the “primed” position will activate the switch previously descried for a second time or alternatively activate contact point 110 of antenna sleeve 11 shown in FIG. 3. The end housing 9 of the injection pen 2 includes an index rib 41 for guiding the button 10. A protrusion 42 of the button 10 moves in a helical cut-out 101 of release ring 28, helping to rotate the release ring 28 to its open position. The button 10 incorporates a cut-out 43 which prevents the button from rotating and accidentally releasing the plunger 13. The helical cut-out 101 in release ring 28 provides a mechanism for rotating release ring 28 into the opening position, releasing plunger rod 24 and subsequently allowing plunger 13 to move forward. The face 102 of the plunger rod 24 is engaged with the face 103 of the release ring 28. The face 102 of plunger rod 24 and face 103 of release ring 28 are only disengaged after the button 10 is pressed and the release ring 28 is rotated to the opening position and lug 27 of the plunger rod 24 can move through cut-out 106 of release ring 28. Release ring 28 is kept in place axially with wings 300 which are clamped between antenna sleeve 11 and end housing 9.

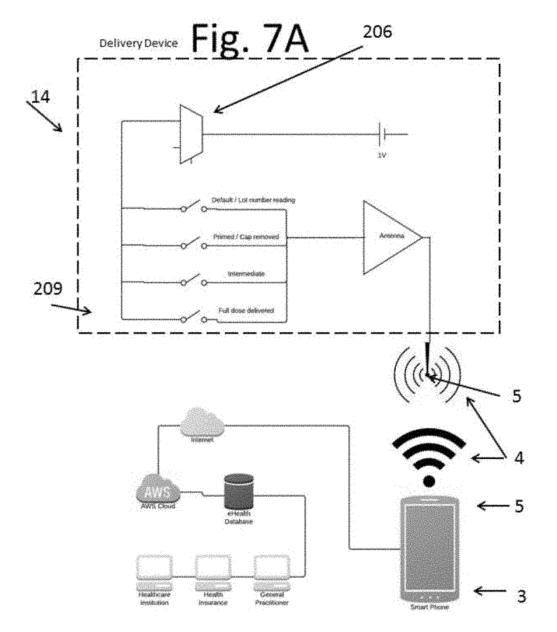

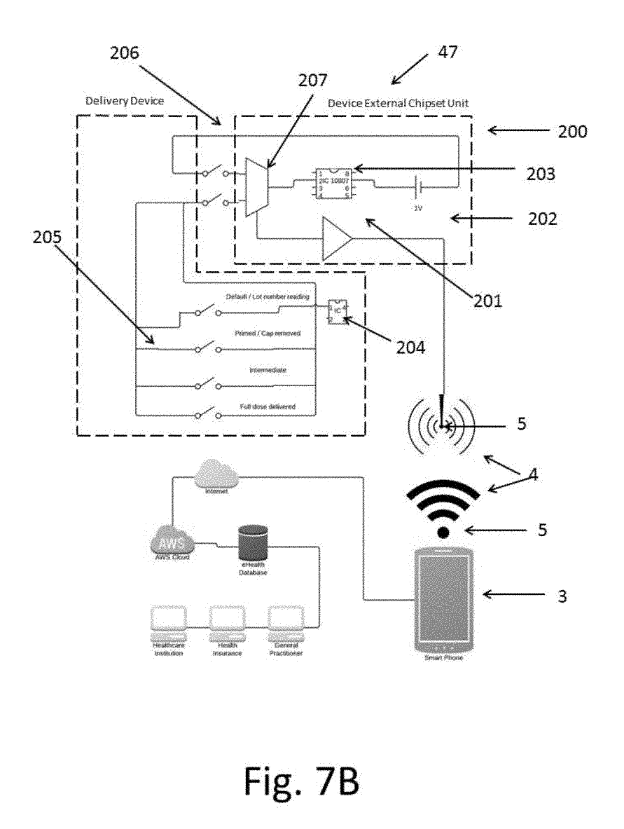

FIG. 7 shows a schematic diagram of injection system with external chipset unit 200 and injection pen with integrated chipset 208 and integrated switch 209. Chipset unit 200 comprises of at least an antenna 201, connection pins 207 on-board contact points 206, power source (battery) 202, wireless communication stack and may comprise of a microprocessor 203. The injection pen shown in the left comprises of a device integrated switch 205 and may comprise of a chipset 204 (RFID chip) holding information such as drug name, lot number, expiry date etc. The chipset 200 may also include a NFC chip which could be read by a telecommunication device.

Computer Software

The invention includes a microprocessor 6 programmed with computer software preferably used with a telecommunication device, to initiate and control the transmission of communication waves between the injection pen 2 and the mobile communication device 3. Modern mobile telephones conveniently operate by computer software integrated into their operating systems. The invention includes programs integrated into the operating system platform of a mobile to transmit and receive data from the operation of the injection pen 2. Preferably the computer software receives and stores signals from the injection pen indicating the status and progress of the injection process, including, for example, priming of the primary drug container, starting the injection of fluid into a subject, ending of the injection process, time of the injection process, and other relevant information. The computer software may include routines which prompt a user to make an injection. The prompt may include an audible signal to alert the user to commence an injection or alternatively the user may receive a message via the mobile devices' messenger system. The injection software may be downloadable from the Internet onto the mobile communication device 3. The injection may be modifiable and updatable to include changes in the aspects such as the timing of prompts to a user to make an injection. Preferable to computer software provides a routine to record injection locations so that changing injecting location can be carried out more accurately. Preferably, the algorithm is programmed in a manner that subsequent signal or data packages received from the pen trigger different prompts and message within the application. Preferably, the software is programmed enabling the patient to use the device without needing a printed instruction for use. The software provides feedback forms enabling the user to enter user feedback at any given time. The software may have different predefined modes allowing the user to select an appropriate mode depending on his preference and experience with the injection system. In example, in the beginning of a treatment the user may wish to be guided trough step by step whereas once the user is familiar with the system he may wish changing to a simpler mode, only using certain functions, in example disabling feedback forms and prompts etc.

Communications

The injection pen 2 is in communication with the mobile communication device 3 with suitable communication means 4. Preferably, the communication is by radio waves emitted and received 4 by antennae 5 located on each of the mobile communication device 3 and the injection pen 2. Preferably the mobile communication device is spaced from the injection pen 2 in order to achieve an ergonomic user experience, ideally placing the phone on a flat surface, such as a table, giving the user am idea view of the screen. Preferably, the mobile communication device 3 is a mobile telephone which has an antenna for radio communication. Alternatively, the mobile communication device 3 may be a watch or other device which is capable of housing a processor and antenna for receiving and transmitting communications with the injection pen. The mobile communication device 3 may be any suitable embodiment such as a watch or mobile telephone. When the mobile communication device 3 is a mobile telephone this most conveniently allows the computer software monitoring and guiding the injection process to be conveniently interfaced with the telephone as described above. Preferably, the radio waves are Radio Frequency waves generated by an electronic chipset 14 or 118 engaged with a housing of the injection pen. The RF technology most advantageously enables communication of low-power radio waves over short distances, preferably 10 cm to 100 cm, such as when an injection pen is carried with a mobile device in a purse, a briefcase, or the like, which allows communication between the injection pen and a mobile communication device while an injection is made. Ideally the mobile phone is placed on a flat surface (table) so that the user has both hands available for giving the injection and so that the screen of the mobile phone can conveniently be viewed. Most preferably, the electronic chipset employs Bluetooth low energy technology. The electronic chipset may operate as a Bluetooth beacon as known in the art. The electronic chipset chip may be powered by a battery 112. Alternatively, it may be powered by the radio waves emitted by the mobile communication device 3, known as passive RFID chips. Alternatively near field communication (NFC) is to communicate with an unpowered chip (passive chip).

In embodiments using a passive chip and powering the device via antenna, the injection pen antenna 5 preferably takes the shape of a coil as illustrated in FIG. 3. However, other shapes are possible, within the range of shapes used for antennae. A passive chip (unpowered) may require a larger antenna whereas with a powered chipset the antenna can be much smaller and integrated in the electronic chipset 14 or 200. The coil configuration as shown in FIG. 3 most conveniently enables the antenna 5 to be coiled around a sleeve 11 within the injection pen housing 8. Preferably, the antenna 5 is coiled in spiral configuration within a spiral groove 36. Alternatively, the antenna may be associated with or adjacent the chipset.

Generating Signals

The antenna 5 is activated to send signals 4 by the movement of contact points or switches 32, 33, 34 on a contact/switch plate 31 spaced along the axis of the injection pen 2. When the contact points pass and contact the switching plate 31, the antenna 5 is energised and transmits a signal 4 which can be received by the antenna 5 of the mobile communication device 3 for processing. In the embodiment shown in the figures, there are switches to indicate and send a signal, when pen is in its default position 32, when the pre-filled needle is primed 33 for injection, when the injection commences, and when the injection is complete 34. However, the number of switches can be increased to monitor more steps in the injection process, such as intermediate steps when only a portion of the fluid in the pre-filled syringe is injected. As described above, the signals are received by the antenna of mobile communication device, which incorporates computer software to record the signals and identify them according to the sequence of transmissions.

Preferably the signals may be generated by a device integrated switch, partly shown in FIG. 3, where two switching plates 106 inserted in antenna sleeve 4 parallel to each other provide the first part of a switch. Contact is made as plunger 13 moves forward with contact plate 107 of (alternative) plunger 108. Contact plate 107 of plunger 108 makes contact with contact points 109, 110 and 111. In example, contact point 111 generates the default signal, contact point 110 generates the priming signal and contact point 109 indicates end of dose delivery. When the contact points pass and contact the switching plate 106 a signal is generated, fed into the electronic chipset and processed by the electronic chipset and transmitted to the mobile communication device 3.

An embodiment of an injection pen 2 incorporating a plunger 13 and an electronic chipset 14 at the distal end is shown in FIG. 4. FIG. 4A shows the location of the chipset 14 and FIG. 4B shows the chipset 14 enlarged and in greater detail. In some embodiments, a battery may be located adjacent the chipset 14 to provide power to the chipset. FIG. 5 shows in planar view an embodiment of a battery casing 37 engaged with chipset 14. A suitable battery can be slotted into the battery recess 38 of the casing to provide power for the chipset in embodiments which include battery power for the chipset.

Alternatively, in one embodiment of the invention, the chipset and battery may be disposed within a housing 118 that may be disengaged from the injection pen as illustrated in FIG. 5. The external chipset preferably includes a battery 112, a microprocessor 113 and on-board contact points 114. This embodiment most conveniently enables separate disposal of the injection pen and external chipset unit after one or a few uses. In this embodiment the external battery housing 118 is also reversibly engageable with the mobile communications device. This embodiment most conveniently saves costs associated with an injection pen that is needed for only one injection but the relatively expensive battery and chipset can be reused.

Power Source

The invention includes that the power for generating signals may be supplied actively from a power source such as a battery 112 incorporated into the chipset housing, or passively from the mobile communication device via the antenna and chipset 14. Alternatively, the minimal power needed for transmitting the signals etc. may be generated by energy harvesting known in the art. The power source can be system integrated as shown FIG. 4E or externally placed shown in electrical unit 118 shown in FIG. 5. In embodiments with an external unit 118, the power source may be rechargeable through the telecommunication device when unit 118 is inserted in housing 46 of casing 400. The external electronic unit 118, including power source 112, conveniently allows separately disposal of the injection pen and re-use of or disposing of the unit 118.

Application Process

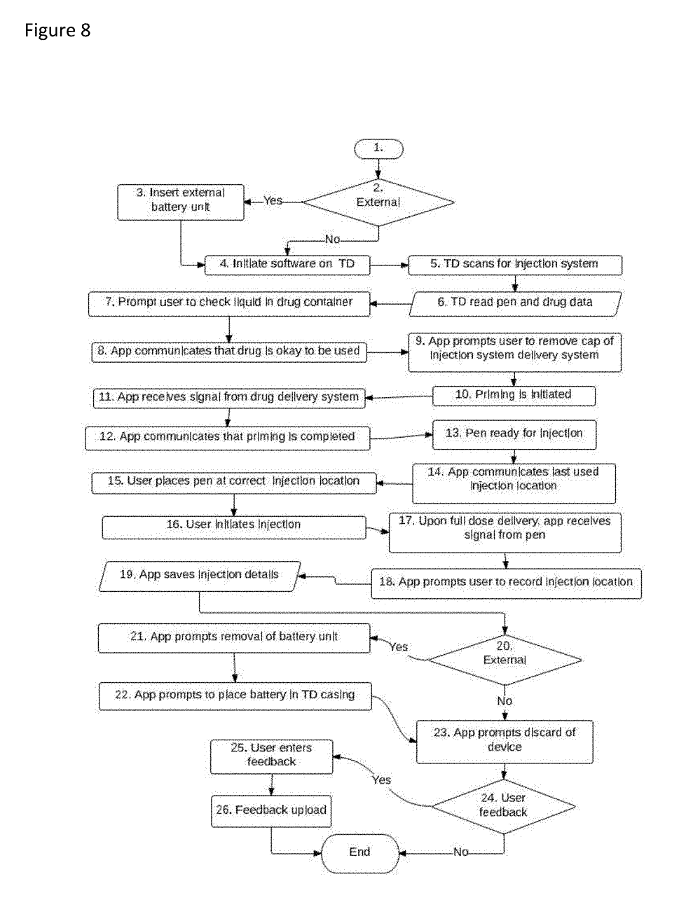

An embodiment of the invention includes the following steps for monitoring and controlling the process of injecting fluids using the injection pen system of the invention as illustrated in FIG. 8. It will be understood that many variations of the steps may be used while practising the invention in different embodiments

The process starts as shown in Box 1. For embodiments which include external battery engagement, this occurs at this step at shown in Box 2 and Box 3. If there is no external battery engagement, or after the external battery is engaged with the injection pen (Box 3), the software for monitoring the injection process is initiated on the telecommunication device (TD) as shown in Box 4, either manually or automatically. The TD then scans for injection system signals as shown in Box 5. The data from the injection pen and relating to the drug therein may be stored in the TD program as shown in Box 6. The user may then be prompted to check liquid in the drug container as shown in Box 7. The computer software may communicate that the drug in the drug container is suitable for use by the user after the system has automatically checked the drug's validity online as shown in Box 8. If so, the computer software prompts the user to remove the cap of the injection pen system as shown in Box 9. Once the cap is removed, the priming step is initiated as shown in Box 10. In the next step signals are generated by the injection pen and transmitted to the TD as shown in Box 11. The computer software may then determine from the signals when the priming step is complete as shown in Box 12. At this point the injection pen is ready to commence the injection as shown in Box 13. In some embodiments where the injection pen is used multiple times, the computer software may store information about where the last injection was made on the body of the user and communicate this this to the user as shown in Box 14. In these embodiments, the user then positions the injection pen at that body location as shown in Box 15. Once placed at an injection position, the user initiates the injection as shown in Box 16. Once the injection has resulted in the delivery of the required dose of fluid, the computer software receives the signal indicating this from the injection pen as shown in Box 17. In some embodiments, the computer software may then prompt the user to record the injection location as shown in box 18. This new data may be saved by the computer program as shown in Box 19. In embodiments where there is external battery management (Box 20), the computer program may prompt the user to remove the battery unit as shown in Box 21, or in embodiments where the injection pen is designed for a single use, the computer program my prompt the user to discard the pen as shown in Box 23. Where the injection pen is designed for multiple uses by a user and it incorporates a removable battery unit for power the computer software may prompt the user to place the battery in the TD casing as shown in Box 22, after which the computer software may prompt the user to discard the injection device as shown in Box 23. In some embodiments, the computer program may incorporate the ability to prompt the user for feedback as shown in Box 24, where the user enters the feedback into the computer program as shown in Box 25. This information may then be uploaded as shown in Box 26. The uploading step may include transfer of the data using the internet. After the completion of the injection, which may include the steps of inputting and uploading feedback from the user, the process ends. The software may have different predefined modes allowing the user to select an appropriate mode depending on his preference and experience with the injection system. For example, in the beginning of a treatment the user may wish to be guided trough step by step whereas once the user is familiar with the system he may wish changing to a simpler mode, only using certain functions, in example disabling feedback forms and prompts etc.

I claim:

1. An injection pen comprising of:
a housing for a syringe having a plunger;
a cap;
a plunger rod for activating the plunger; and
a communication means for communicating the displacement of the syringe plunger, said communication means reversibly engageable with the injection pen.

2. An injection system comprising of:
an injection pen comprising of a housing for a syringe;
a syringe;
means for operating the plunger of said syringe;
a removable communication means for transmitting and receiving signals; and
a microprocessor programmed with computer software for monitoring and guiding an injection from a syringe;
wherein said programmed microprocessor receives signal data from said communication means.

3. The injection pen of claim 1 further comprising of a triggering means to trigger injection.

4. The injection pen of claim 3 wherein said triggering means includes a button.

5. The injection system of claim 2 wherein the programmed microprocessor is spaced from said injection pen.

6. The injection system of claim 5 wherein the programmed microprocessor is incorporated into a mobile phone.

7. The injection system of claim 6 wherein the computer software displays the output of said monitoring on the screen of said mobile phone.

8. The injection system of claim 2 wherein said transmitted signals initiate said computer software for monitoring and guiding said injection.

9. The injection system of claim 2 wherein the communication means includes an antenna.

10. The injection system of claim 2 wherein the signal data is transmitted by an RFID chipset.

11. The injection system of claim 10 wherein said RFID chipset is received into a housing connected to a mobile phone.

12. The injection system of claim 11 further comprising of a power source.

13. The injection system of claim 12 wherein the power source is a battery.

14. The injection system of claim 11 further comprising of a power source and communication means that are reversibly engageable.

15. The injection pen of claim 1 further comprising of a power source.

16. The injection pen of claim 15 wherein the power source is a battery.
